(19) United States
(12) Patent Application Publication
(10) Pub. No.: US 2023/0248674 A1
(43) Pub. Date: Aug. 10, 2023

(54) METHODS OF INTRAVENOUSLY ADMINISTERING SOTALOL HYDROCHLORIDE

(71) Applicant: AltaThera Pharmaceuticals LLC, Chicago, IL (US)

(72) Inventor: Brandon Ira Kashfian, Chicago, IL (US)

(21) Appl. No.: 18/135,467

(22) Filed: Apr. 17, 2023

Related U.S. Application Data
(63) Continuation-in-part of application No. 18/126,561, filed on Mar. 27, 2023.
Application No. 18/126,561 is a continuation-in-part of application No. 17/585,190, filed on Jan. 26, 2022.
Application No. 17/585,190 is a continuation of application No. 16/863,567, filed on Apr. 30, 2020, now abandoned.
Application No. 16/863,567 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/863,567 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
Application No. 18/126,561 is a continuation-in-part of application No. 17/306,490, filed on May 3, 2021.
Application No. 17/306,490 is a continuation of application No. 16/849,099, filed on Apr. 15, 2020, now abandoned.
Application No. 16/849,099 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 17/306,490 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
Application No. 16/103,815 is a continuation-in-part of application No. 17/585,190, filed on Jan. 26, 2022.
Application No. 17/585,190 is a continuation of application No. 16/863,567, filed on Apr. 30, 2020, now abandoned.
Application No. 16/863,567 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/863,567 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
Application No. 16/103,815 is a continuation-in-part of application No. 17/306,490, filed on May 3, 2021.
Application No. 17/306,490 is a continuation of application No. 16/849,099, filed on Apr. 15, 2020, now abandoned.
Application No. 16/849,099 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 17/306,490 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
Application No. 17/306,490 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
(60) Provisional application No. 63/009,511, filed on Apr. 14, 2020.
(60) Provisional application No. 62/987,832, filed on Mar. 10, 2020.
(60) Provisional application No. 63/346,003, filed on May 26, 2022.
(60) Provisional application No. 63/332,882, filed on Apr. 20, 2022.
(60) Provisional application No. 63/331,467, filed on Apr. 15, 2022.
(60) Provisional application No. 63/323,836, filed on Mar. 25, 2022.
(60) Provisional application No. 63/346,003, filed on May 26, 2022.
(60) Provisional application No. 63/332,882, filed on Apr. 20, 2022.
(60) Provisional application No. 63/331,467, filed on Apr. 15, 2022.
(60) Provisional application No. 63/009,511, filed on Apr. 14, 2020.
(60) Provisional application No. 62/987,832, filed on Mar. 10, 2020.

(51) Int. Cl. A61K 31/18; A61K 9/00; A61P 9/06

(52) U.S. Cl. CPC A61K 31/18; A61K 9/0019; A61P 9/06

(57) ABSTRACT

Methods of administering sotalol hydrochloride in an amount effective for treating a cardiovascular condition are described. An initial IV loading dose can be administered over a period of about one hour. The subject/patient can be discharged from the medical facility providing cardiac monitoring prior to administration of oral doses. The IV can be administered in a manner such that maximum serum concentration of sotalol is reached and the subject/patient can be discharged before administration of a first oral dose.

### CROSS REFERENCE TO RELATED APPLICATIONS

The present application is a Continuation in Part (CIP) application of U.S. application Ser. No. 18/126,561, filed Mar. 27, 2023. The '561 application is a Continuation in Part (CIP) application of U.S. application Ser. No. 17/585,190, filed Jan. 26, 2022, which application is a Continuation application of U.S. application Ser. No. 16/863,567, filed Apr. 30, 2020. The '567 application relies on the disclosure of and claims priority to and the benefit of the filing date of U.S. Provisional Application No. 63/009,511, filed Apr. 14, 2020 and is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '567 application is a CIP application of U.S. application Ser. No. 16/693,310, filed Nov. 24, 2019, which application is a CIP application of U.S. application Ser. No. 16/103,815, filed Aug. 14, 2018. The '561 application is a CIP application of U.S. application Ser. No. 17/306,490, filed May 3, 2021, which application is a Continuation application of U.S. application Ser. No. 16/849,099, filed Apr. 15, 2020. The '099 application relies on the disclosure of and claims priority to and the benefit of the filing date of U.S. Provisional Application No. 62/987,832, filed Mar. 10, 2020. The '490 application is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '490 application is a CIP application of U.S. application Ser. No. 16/693,310, filed Nov. 24, 2019, which application is a CIP application of the '815 application. The '561 application further relies on the disclosure of and claims priority to and the benefit of the filing date of each of U.S. Provisional Application Nos. 63/346,003, filed May 26, 2022, 63/332,882, filed Apr. 20, 2022, 63/331,467, filed Apr. 15, 2022, and 63/323,836, filed Mar. 25, 2022. The present application is a Continuation in Part (CIP) application of U.S. application Ser. No. 17/585,190, filed Jan. 26, 2022, which application is a Continuation application of U.S. application Ser. No. 16/863,567, filed Apr. 30, 2020. The '567 application relies on the disclosure of and claims priority to and the benefit of the filing date of U.S. Provisional Application No. 63/009,511, filed Apr. 14, 2020 and is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '567 application is a CIP application of U.S. application Ser. No. 16/693,310, filed Nov. 24, 2019, which application is a CIP application of U.S. application Ser. No. 16/103,815, filed Aug. 14, 2018. The present application is a CIP application of U.S. application Ser. No. 17/306,490, filed May 3, 2021, which application is a Continuation application of U.S. application Ser. No. 16/849,099, filed Apr. 15, 2020. The '099 application relies on the disclosure of and claims priority to and the benefit of the filing date of U.S. Provisional Application No. 62/987,832, filed Mar. 10, 2020. The '490 application is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '490 application is a CIP application of U.S. application Ser. No. 16/693,310, filed Nov. 24, 2019, which application is a CIP application of the '815 application. The present application further relies on the disclosure of and claims priority to and the benefit of the filing date of each of U.S. Provisional Application Nos. 63/346,003, filed May 26, 2022, 63/332,882, filed Apr. 20, 2022, and 63/331,467, filed Apr. 15, 2022. The disclosures of these applications are hereby incorporated by reference herein in their entireties.

### BACKGROUND OF THE INVENTION

### Field of the Invention

The present invention relates to the field of cardiovascular pharmaceutics, more particularly compositions and methods for the treatment of cardiovascular conditions, such as arrhythmias, with intravenous anti-arrhythmics, such as sotalol hydrochloride.

### Description of Related Art

Sotalol hydrochloride is a racemic mixture of d- and 1-sotalol hydrochloride. Sotalol hydrochloride is a white, crystalline solid with a molecular weight of 308.8. It is hydrophilic, soluble in water, propylene glycol, and ethanol. Chemically, sotalol hydrochloride is a d,l-N-[4-[1-hydroxy-2-[(1-methylethyl)amino]ethyl]phenyl]methane-sulfonamide monohydrochloride. (HIGHLIGHTS OF PRESCRIBING INFORMATION: Sotalol hydrochloride injection for intravenous use, Revised Jul. 2009). The molecular formula is Cl2H2ON2O3S·HCl and is represented by the following structural formula:

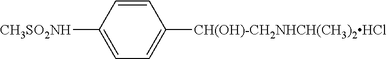

Intravenous sotalol is supplied as a sterile, clear solution in 10 ml vials, each vial containing 150 mg sotalol hydrochloride in acetate buffer. Sotalol hydrochloride is an antiarrhythmic drug with both beta adrenoreceptor blocking (Class II) and cardiac action potential duration prolongation (Class III) properties. The drug is hemodynamically well-tolerated and has a small risk of proarrhythmia. Both isomers have similar Class III activity, while the 1-isomer is responsible for virtually all of the beta blocking activity. Sotalol does not have partial beta agonist or membrane stabilizing activity (Na channel inhibition). Sotalol does not undergo metabolism and is nearly 100% bioavailable when taken on an empty stomach. Sotalol hydrochloride does not bind to plasma proteins. The pharmacokinetics of d- and 1-sotalol enantiomers are essentially identical. Excretion is predominantly via the kidney in the unchanged form and therefore lower doses are necessary in patients with renal impairment.

Sotalol is FDA approved for the maintenance of normal sinus rhythm in patients with history of highly symptomatic atrial fibrillation/flutter and for the treatment of documented life-threatening ventricular arrhythmias. Sotalol is currently approved in the US for oral administration (for example, under the brand name BETAPACE AF®, Bayer HealthCare Pharmaceuticals Inc.) and is approved for IV administration (AltaThera Pharmaceuticals LLC). Sotalol is an effective antiarrhythmic agent but also can cause serious ventricular arrhythmias, primarily Torsade de Pointes (TdP) type ventricular tachycardia, a polymorphic ventricular tachycardia associated with QTc prolongation. QTc prolongation is directly related to the plasma concentration of sotalol. Conventionally, steady-state plasma levels of sotalol and maximum QTc prolongation are obtained in 3 days with oral administration (i.e. after 5-6 doses when administered twice daily). QTc changes are directly related to maximum serum concentration of the sotalol. To minimize the risk of sotalol caused arrhythmias, the product label mandates that patients initiated or re-initiated on sotalol should be hospitalized for at least three days or until steady state drug levels are achieved, in a facility that can provide cardiac resuscitation and continuous electrocardiographic monitoring.

Hospitalization represents a high-cost burden in the United States. The three-day hospital stay is also burdensome to the patient, resulting in loss of productivity, time away from family, and an increased risk of hospital-acquired infections, as well as a diversion of hospital resources, staff, and patient care beds. The availability of IV sotalol offers an opportunity to improve on the sotalol loading protocol and decreases the length of hospital stay.

### SUMMARY OF THE INVENTION

Sotalol is a commonly used oral anti-arrhythmic drug for management of atrial arrhythmias. Due to a risk of pro-arrhythmia, inpatient initiation of sotalol with QTc monitoring for five doses (a typical three-day protocol) is a standard treatment. Patent literature relating to sotalol includes: U.S. Pat. Nos. 10,512,620 and 10,799,138; and US Published Patent Application Nos. 2020/0093759, 20200085771, 2021/0283049, 20200253903, 20200338027, 2019/0307343, 2019/0380605, 2021/0076959, which are incorporated by reference herein in their entireties.

This disclosure provides a change from the guidelines for sotalol in-hospital loading that achieves steady-state blood level and maximum QTc prolongation within hours, such as within 1 hour. Pharmacokinetic/pharmacodynamics (PK/PD) simulations indicate that administering an intravenous loading dose of sotalol can achieve steady state Cₘₐₓ and thus maximum QTc prolongation within hours, such as within 1 hour. Thus, the present methods can enable physicians to evaluate the major safety concern, excessive QTc prolongation, in hours instead of days.

Aspects of embodiments of the invention include the following Aspects:

Aspect 1 is a method of administering sotalol hydrochloride therapy using a single intravenous (IV) dosage, the method comprising: (1) identifying a subject as having symptomatic atrial fibrillation or atrial flutter, and currently in sinus rhythm; (2) administering a single IV dosage of sotalol hydrochloride, for a period of 1 hour, to the subject who is also in a facility capable of providing cardiac resuscitation and continuous electrocardiographic monitoring, wherein the single IV dosage is administered: (a) in an amount of about 55-128 mg, for the subject who is naïve to sotalol; (b) in an amount of about 73.8-105.6 mg, for the subject who had received 80 mg of oral sotalol hydrochloride prior to the IV dosage; or (c) in an amount of about 88-140.8 mg, for the subject who had received 120 mg of oral sotalol hydrochloride prior to the IV dosage; (3) after being discharged from the facility capable of providing cardiac resuscitation and continuous electrocardiographic monitoring, and starting at least about 2 hours after completion of the single IV dosage, such as at about 4 hours after completion of the single IV dosage, administering an oral dosage of sotalol hydrochloride selected from: (a) 80 mg or 120 mg after completion of the (2)(a) IV dosage; or (b) 120 mg after completion of the (2)(b) IV dosage; or (c) 160 mg after completion of the (2)(c) IV dosage; and (4) optionally repeating oral administration of sotalol hydrochloride at least once at a selected interval.

Aspect 2 is a method, comprising: administering sotalol hydrochloride to a patient; wherein the sotalol hydrochloride is administered: as a loading dose, intravenously, over a period of about 1 hour and in an amount of 49.5 mg to 140.8 mg; as a first oral dose, in an amount of 80 mg, 120 mg, or 160 mg; and as a subsequent oral dose, in an amount of 80 mg, 120 mg, or 160 mg.

Aspect 3 is a method of initiating or escalating sotalol hydrochloride treatment in a patient comprising: (A) determining a creatinine clearance of the patient; (B) determining a QTc interval of the patient; (C) administering to the patient an IV loading dose of sotalol hydrochloride, wherein the IV loading dose is selected from an amount ranging from 49.5 mg to 140.8 mg, and wherein optionally the IV loading dose is administered over about 60 minutes, optionally by way of several IV doses; (D) determining a second QTc interval of the patient; and (E) providing a prescription for administering oral sotalol hydrochloride to the patient in a manner such that: a first oral dose of about 80 mg, 120 mg, or 160 mg is to be administered about 2-12 hours after completion of the IV loading dose; and one or more subsequent oral dose(s) of about 80 mg, 120 mg, or 160 mg is to be administered at about 12 hour, 24 hour, or 48 hour interval(s), beginning about 12 hours, 24 hours, or 48 hours after administration of the first oral dose, wherein optionally the IV dosage is administered to the patient while admitted to a facility capable of providing cardiac resuscitation and continuous electrocardiographic monitoring and the first oral dose and any subsequent oral doses are administered to the patient after the patient has been discharged from such facility.

Aspect 4 is a method of initiating or escalating sotalol hydrochloride treatment in a patient having symptomatic atrial fibrillation or atrial flutter, and currently in sinus rhythm, comprising: (A) determining a creatinine clearance of the patient; (B) determining a QTc interval of the patient; (C) administering to the patient an IV loading dose of sotalol hydrochloride, wherein the IV loading dose is chosen from an amount ranging from: i. 55-63 mg, such as 60 mg, wherein the patient is being initiated for a target oral dose of 80 mg, and the creatinine clearance of the patient is >90 mL/min; ii. 73.8 mg to 88 mg, such as 75 mg or 82.5 mg, wherein the patient is being initiated for a target oral dose of 80 mg, and the creatinine clearance of the patient is 10 mL/min to 90 mL/min; iii. 70-80 mg, such as 75 mg, wherein the patient is being is being escalated to a target oral dose of 120 mg, and the creatinine clearance of the patient is >90 mL/min; iv. 82-88 mg, such as 82.5 mg, wherein the patient is being is being escalated to a target oral dose of 120 mg, and the creatinine clearance of the patient is 10 mL/min to 90 mL/min; v. 82 mg to 96 mg, such as 90 mg, wherein the patient is being initiated for a target oral dose of 120 mg, or is being escalated to a target oral dose of 160 mg, and the creatinine clearance of the patient is >90 mL/min; or vi. 99 mg to 140.8 mg, such as 105 mg, 112.5 mg or 125 mg, wherein the patient is being initiated for a target oral dose of 120 mg, or is being escalated to a target oral dose of 160 mg, and the creatinine clearance of the patient is 10 to 90 mL/min; (D) determining a second QTc interval of the patient; and (E) providing a prescription for administering oral sotalol hydrochloride to the patient in a manner such that one or more oral dose(s) of the sotalol hydrochloride is to be administered after completion of the IV loading dose, wherein optionally the IV dosage is administered to the patient while admitted to a facility capable of providing cardiac resuscitation and continuous electrocardiographic monitoring and the first oral dose and any subsequent oral doses are administered to the patient after the patient has been discharged from such facility.

Aspect 5 is a method of any of Aspects 1-4, further comprising administering a second oral dose of sotalol hydrochloride to the patient 12-48 hours after the first oral dose.

Aspect 6 is the method of any of Aspects 1-5, further comprising administering one or more subsequent oral dose(s) of sotalol hydrochloride to the patient at an interval of 12-48 hours.

Aspect 7 is the method of any of Aspects 1-6, wherein the first oral dose is administered about 4 hours after completion of the administration of the IV loading dose.

Aspect 8 is the method of any of Aspects 1-7, wherein the IV loading dose is administered in an amount of about 55 to 73.8 mg.

Aspect 9 is the method of any of Aspects 1-8, wherein the IV loading dose is administered in an amount of about 70 to 88 mg.

Aspect 10 is the method of any of Aspects 1-9, wherein the IV loading dose is administered in an amount of about 80 mg to 105.6 mg.

Aspect 11 is the method of any of Aspects 1-10, wherein the IV loading dose is administered in an amount of 99 mg to 140.8 mg.

Aspect 12 is the method of any of Aspects 1-11, wherein the patient had received a prior dose of sotalol hydrochloride 12-24 hours prior to administration of the IV loading dose.

Aspect 13 is the method of any of Aspects 1-12, wherein the patient is capable of experiencing a sotalol hydrochloride Cₘₐₓ steady state within about 8 hours of administration of the IV loading dose.

Aspect 14 is the method of any of Aspects 2-13, wherein the IV loading dose is administered to the patient in a hospital or facility capable of providing cardiac resuscitation and continuous ECG monitoring.

Aspect 15 is the method of any of Aspects 2-14, wherein the first oral dose and any subsequent oral dose(s) are administered after the patient is discharged from the hospital or facility capable of providing cardiac resuscitation and continuous ECG monitoring.

Aspect 16 is the method of any of Aspects 1-15, wherein the patient has a creatinine clearance of >90 mL/min.

Aspect 17 is the method of any of Aspects 1-16, wherein the patient has a creatinine clearance of >60 mL/min to 90 mL/min.

Aspect 18 is the method of any of Aspects 1-17, wherein the patient has a creatinine clearance of >30 mL/min to 60 mL/min.

Aspect 19 is the method of any of Aspects 1-18, wherein the patient has a creatinine clearance of 10 mL/min to 30 mL/min.

Aspect 20 is the method of any of Aspects 1-19, further comprising measuring a QT or QTc interval of the patient before administration of the IV loading dose.

Aspect 21 is the method of any of Aspects 1-20, further comprising measuring a QT or QTc interval of the patient after administration of the IV loading dose, but before administration of the first oral dose.

Aspect 22 is the method of any of Aspects 1-21, further comprising measuring a QT or QTc interval of the patient after administration of the second oral dose.

Aspect 23 is the method of any of Aspects 1-22, wherein the patient has a cardiovascular condition.

Aspect 24 is the method of any of Aspects 1-23, wherein the cardiovascular condition is selected from atrial fibrillation, atrial flutter, ventricular tachycardia, hemodynamically stable or unstable ventricular tachycardia, paroxysmal atrial fibrillation, ventricular fibrillation, paroxysmal supraventricular tachycardia, heart failure, coronary artery disease, and pulmonary artery hypertension.

Aspect 25 is the method of any of Aspects 1-24, wherein the first oral dose amount is based on one or more QT or QTc interval.

Aspect 26 is the method of any of Aspects 1-25, wherein the second oral dose amount is different from the first oral dose amount.

Aspect 27 is the method of any of Aspects 1-26, wherein the patient is being treated for atrial fibrillation and/or atrial flutter.

Aspect 28 is the method of any of Aspects 1-27, wherein the patient is currently in normal sinus rhythm.

Aspect 29 is the method of any of Aspects 1-28, wherein the IV loading dose is administered to the patient in an amount and over a period of time such that the sotalol hydrochloride reaches or is capable of reaching Cₘₐₓ steady state in the patient during the administering of the IV loading dose or within 2 hours of the administering of the IV loading dose.

Aspect 30 is the method of any of Aspects 1-29, wherein the IV loading dose is administered to the patient in an amount and over a period of time such that the sotalol hydrochloride reaches or is capable of reaching a Cₘₐₓ in the patient that is at least about 85% of a steady state Cₘₐₓ for an oral dosing protocol of 80 mg, 120 mg, or 160 mg sotalol hydrochloride, such as about 87%, 90%, 92%, 95%, 97% or 99%.

Aspect 31 is a method of administering therapy using a single intravenous (IV) dosage of sotalol hydrochloride, the method comprising: administering to a subject a single IV dosage of sotalol hydrochloride; wherein the subject is in a facility capable of providing cardiac resuscitation and continuous electrocardiographic monitoring; wherein the single IV dosage is administered for a period of 1 hour and in an amount in the range of about 49.5-128 mg; and at least about 1 hour after completion of the single IV dosage, and after being discharged from the facility capable of providing cardiac resuscitation and continuous electrocardiographic monitoring, administering an oral dosage of sotalol hydrochloride selected from 80 mg, 120 mg or 160 mg.

Aspect 32 is a method of initiating or escalating sotalol hydrochloride treatment in a subject comprising: (A) determining a creatinine clearance of the subject; (B) determining a QTc interval of the subject; (C) administering to the subject an IV loading dose of sotalol hydrochloride, wherein the IV loading dose is selected from an amount in the range of from about 49.5 mg to 141 mg; (D) determining a second QTc interval of the subject; and (E) providing a prescription for administering oral sotalol hydrochloride to the subject in a manner such that: a first oral dose of about 80 mg, 120 mg, or 160 mg is to be administered to the subject about 2-12 hours after completion of the IV loading dose; and one or more subsequent oral dose(s) of about 80 mg, 120 mg, or 160 mg is to be administered to the subject at about 12 hour, 24 hour, or 48 hour interval(s), beginning about 12 hours, 24 hours, or 48 hours after administration of the first oral dose, wherein optionally the IV dosage is administered to the subject while admitted to a facility capable of providing cardiac resuscitation and continuous electrocardiographic monitoring and the first oral dose and any subsequent oral doses are administered to the subject after the patient has been discharged from such facility.

Aspect 33 is the method of Aspect 31 or 32, wherein: the IV loading dose comprises an amount of sotalol hydrochloride in the range of from about 88.2 mg to 96 mg; the IV loading dose is administered to the subject over a period of 1 hour; the subject has normal kidney function and a creatinine clearance of >90 mL/min; and the oral dosage of sotalol hydrochloride is 160 mg.

Aspect 34 is the method of Aspect 31 or 32, wherein: the IV loading dose comprises an amount of sotalol hydrochloride in the range of from about 82 mg to 88 mg; the IV loading dose is administered to the subject over a period of 1 hour; and the oral dosage of sotalol hydrochloride is 80 mg.

Aspect 35 is the method of Aspect 31 or 32, wherein: the IV loading dose comprises an amount of sotalol hydrochloride in the range of from about 110 mg to 128 mg; the IV loading dose is administered to the subject over a period of 1 hour; the subject is renally impaired and has a creatinine clearance of 10-30 mL/min; and the oral dosage of sotalol hydrochloride is 120 mg.

Aspect 36 is the method of Aspect 31 or 32, wherein: the IV loading dose comprises an amount of sotalol hydrochloride in the range of from about 73.8 mg to 82 mg; the IV loading dose is administered to the subject over a period of 1 hour; the subject is renally impaired and has a creatinine clearance of 10-30 mL/min; and the oral dosage of sotalol hydrochloride is 80 mg.

Aspect 37 is the method of Aspect 31 or 32, wherein: the subject is being treated for atrial fibrillation and/or atrial flutter, the subject is currently in normal sinus rhythm, and the subject is naïve to sotalol or has not received sotalol for at least five (5) half-lives of sotalol; and the IV loading dose comprises an amount of sotalol hydrochloride in the range of from about 99 mg to 141 mg.

Aspect 38 is the method of any of Aspects 31-37, wherein the subject is capable of experiencing a sotalol hydrochloride Cₘₐₓ steady state within about 8 hours of administration of the IV loading dose.

Aspect 39 is the method of any of Aspects 31-38, wherein the IV loading dose is administered to the subject in a hospital or facility capable of providing cardiac resuscitation and continuous ECG monitoring.

Aspect 40 is the method of any of Aspects 31-39, wherein the first oral dose and one or more of the subsequent oral dose(s) are administered after the subject is discharged from the hospital or facility capable of providing cardiac resuscitation and continuous ECG monitoring.

Aspect 41 is the method of any of Aspects 31-40, further comprising: measuring a QT or QTc interval of the subject before administering the IV loading dose; measuring a QT or QTc interval of the subject after administering the IV loading dose, but before administering the first oral dose; wherein the QT or QTc interval after administering the IV loading dose is less than a 20% increase from the QT or QTc interval measured before administering the IV loading dose.

Aspect 42 is the method of any of Aspects 31-41, wherein the cardiovascular condition is selected from atrial fibrillation, atrial flutter, ventricular tachycardia, hemodynamically stable or unstable ventricular tachycardia, paroxysmal atrial fibrillation, ventricular fibrillation, paroxysmal supraventricular tachycardia, heart failure, coronary artery disease, and pulmonary artery hypertension.

Aspect 43 is the method of any of Aspects 31-42, wherein the subject is being treated for atrial fibrillation and/or atrial flutter.

Aspect 44 is the method of any of Aspects 31-43, wherein the subject is currently in normal sinus rhythm.

Aspect 45 is the method of any of Aspects 31-44, wherein the IV loading dose is administered to the subject in an amount and over a period of time such that the sotalol hydrochloride reaches or is capable of reaching Cₘₐₓ steady state in the subject during or within 1-2 hours of the administering of the IV loading dose.

Aspect 46 is the method of any of Aspects 31-45, wherein the IV loading dose is administered to the subject in an amount and over a period of time such that the sotalol hydrochloride reaches or is capable of reaching a Cₘₐₓ in the subject that is at least about 85% of a steady state Cₘₐₓ for an oral dosing protocol of 80 mg, 120 mg, or 160 mg sotalol hydrochloride.

### DETAILED DESCRIPTION OF VARIOUS EMBODIMENTS OF THE INVENTION

Reference will now be made in detail to various illustrative implementations. It is to be understood that the following discussion of implementations is not intended to be limiting.

AF is atrial fibrillation.

AFL is atrial flutter.

AF/AFL is atrial fibrillation and/or atrial flutter.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human patient.

BP is blood pressure.

HR is heart rate.

Renally impaired refers to patients having creatinine clearance rates of 60 mL/min, such as ≤30 mL/min.

Escalation means increasing the sotalol dosage of a patient already receiving sotalol, for example where a subject is currently taking a specific amount of an oral dose and escalation involves administering one or more IV doses to escalate the subject to a higher target oral dose.

Sotalol and sotalol hydrochloride (used interchangeably herein) refer to d,l-sotalol hydrochloride.

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's physical or mental well-being. The treatment or amelioration of symptoms can be based on objective or subjective parameters, including the results of a physical examination, neuropsychiatric examinations, or psychiatric evaluation.

Cₘₐₓ ss is the maximal concentration obtained at steady state.

QT is the interval measured from the start of the Q wave or QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment (e.g., during loading or maintenance).

The term “about” used herein in the context of quantitative measurements means the indicated amount ±10%. For example, “about 2 mg” can mean 1.8-2.2 mg.

Hospital refers to a medical facility staffed and equipped to provide continuous ECG monitoring and cardiac resuscitation to patients, if needed. Typically, the medical personnel are trained in the management of serious ventricular arrhythmias.

Reducing or shortening the length of a hospital stay refers to reducing/shortening the length of time a patient is admitted for oral sotalol initiation or escalation, for example at a hospital or other facility capable of providing cardiac resuscitation and continuous electrocardiographic monitoring. For example, a patient would typically require a 3-day (72 hour) stay in such hospital or facility to be initiated/escalated on oral sotalol.

Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm. Sotalol is also indicated for the treatment of life-threatening ventricular tachycardia.

For either indication, intravenous sotalol, when used as a loading dose, achieves steady state concentration faster compared to the conventional oral dosing (e.g., typically 3 days for a non-renally impaired patient).

Typically, IV sotalol is diluted for infusion. For example, IV sotalol can be diluted in saline, 5% dextrose in water (D5W), or Ringer's lactate. The dilution volume chosen is one that is convenient for administration and consistent with fluid restriction. A volumetric infusion pump can be used to administer the IV sotalol.

Typically, other antiarrhythmic therapy is withdrawn prior to starting sotalol.

IV and Oral Dosing

In embodiments, the IV loading dose is delivered by way of an IV infusion over a period of about 1 hour, such as in the range of about 55 minutes to 65 minutes, about 50 minutes to about 70 minutes, or about 45 minutes to about 75 minutes. The IV loading dose is administered while the patient/subject is admitted to a hospital or other facility capable of providing cardiac resuscitation and continuous electrocardiographic monitoring.

In embodiments, the IV loading dose is administered in an amount of about 60 mg, about 75 mg, or about 90 mg, such as about 55 mg to about 65 mg, about 50 mg to about 70 mg, about 70 mg to about 80 mg, about 65 mg to about 85 mg, about 85 mg to about 95 mg, about 80 mg to about 100 mg, or about 45 mg to about 105 mg, or about 110-128 mg, or about 99-140.8 mg or about 49.5-70.2 mg, or about 73.8-105.6 mg, or about 63-88.2 mg, or about 55-64 mg, or about 82-96 mg, or about 49.5-70.4 mg, or about 73.8-105.6 mg, or about 63-88 mg, or about 70-80 mg, or any range in between. The loading dose can also be expressed in mg/min., such as from about 0.825-1.17 mg/min. (49.5-70.2 mg), or from about 1.23-1.76 mg/min. (73.8-105.6 mg), or from about 1.65-2.35 mg/min. (99-140.8 mg), or from about 1.05-1.47 mg/min. (66-88.2 mg).

In embodiments, one or more oral maintenance dose is administered, with a first oral dose being administered about 1 hour to about 6 hours after administration of the IV loading dose, such as about 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, or 5.75 hours after administration of the IV loading dose. In embodiments, the first oral dose is administered about 1 hour to about 12 hours after conclusion of the administration of the loading dose, such as about 2 hours to about 11 hours, about 3 hours to about 10 hours, about 4 hours to about 9 hours, about 5 hours to about 8 hours, or about 6 hours to about 7 hours after conclusion of the IV administration. In embodiments, the subject/patient is administered a first oral dose at least 4 hours after completion of the IV dose. In embodiments, the subject/patient is administered the first oral dose after being released from the hospital or other facility that was providing the monitoring, but the patient/subject can still be monitored in a similar way, such as by using a portable/wearable ECG monitoring system.

In embodiments, the one or more oral dose is selected from about 80 mg, about 120 mg, or about 160 mg. In embodiments, more than one oral dose amount is given to a particular patient (for example, a patient receives one or more oral dose at 120 mg and a subsequent higher oral dose of 160 mg or a subsequent lower oral dose of 80 mg, or any combination thereof).

In embodiments, the IV loading dose and/or one or more of the oral doses is selected based on a patient's creatinine clearance. For example, in some cases a patient/subject with a lower creatinine clearance may receive a lower oral dose than a patient with a higher creatinine clearance, or in some cases a patient/subject with a higher creatinine clearance may receive a lower oral dose than a patient with a lower creatinine clearance. In some cases, for example, the amount of sotalol hydrochloride, whether by IV or an oral dose, appropriate to administer to a subject with a CrCl of 10-30 mL/min or 30-60 mL/min can be higher than is appropriate for a subject with a CrCl of >90 mL/min.

In embodiments, one or more of the oral doses is selected based on a patient's change in QTc (for example, a patient that experiences an increase in QTc of less than 20% after receiving the loading dose may receive a higher oral dose than a patient that experiences an increase in QTc of 20% or greater after receiving the loading dose). In embodiments, the oral dose is changed after administration of a previous oral dose due to a change in the patient's QTc (for example, a patient given an oral dose of 120 mg experiences an increase in QTc of 20% or greater, and the next oral dose can be lowered to 80 mg).

In embodiments, subsequent oral doses are given after the first oral dose. In embodiments, the subsequent oral doses are administered at intervals of about 12 hours, about 24 hours, or about 48 hours. In embodiments, the subject/patient is administered subsequent oral dose(s) after being released from the hospital or other facility capable of providing cardiac resuscitation and continuous electrocardiographic monitoring.

In embodiments, the IV loading dose is administered in a hospital or other facility that can provide cardiac resuscitation and continuous electrocardiographic monitoring and a first oral dose and subsequent oral doses are administered by the patient after discharge from the facility/hospital, but may still be under monitoring, such as self-monitoring.

In embodiments, the IV loading dose and/or maintenance dose(s) are given in amounts and at time intervals such that the patient achieves or is capable of achieving Cₘₐₓ ss within about 7-8 hours, or even within about 1-2 hours, of the start of the administration of the IV loading dose. In embodiments, Cₘₐₓ ss is achieved before administration of the first maintenance dose.

In embodiments, the IV loading dose is given in an amount such that the patient experiences a Cₘₐₓ that is at least about 80% of the Cₘₐₓ ss for the patient's specific dosing protocol within about 2 hours of initiation of the loading dose. In embodiments, following the IV loading dose, such as before the first oral dose, the patient experiences or is capable of experiencing a Cₘₐₓ that is at least about 85% of the Cₘₐₓ ss, such as about 87%, 90%, 92%, 95%, 97% or 99% of the Cₘₐₓ ss for the patient's specific dosing protocol.

In embodiments, the patient receiving sotalol hydrochloride has experienced AF, but is in normal sinus rhythm prior to administration of the sotalol hydrochloride loading dose.

### Example 1

In an embodiment of the invention, sotalol therapy is initiated or escalated by administering to a patient in need thereof an IV loading dose and one or more oral dose of sotalol according to the criteria shown in Table 1.

TABLE 1
IV Sotalol Loading Dosage and Initiation-Escalation Protocol

| | IV loading dose (1 h infusion) | | | | Minimum | |
|---|---|---|---|---|---|---|
| Creatinine | when the oral dose is going from . . . | | | | delay to | Oral |
| Clear- | Sotalol Initiation | | Sotalol Escalation | | first oral | dosing |
| ance* | 0 to | 0 to | 80 to | 120 to | dose | interval |
| (mL/min) | 80 mg** | 120 mg | 120 mg | 160 mg | (h) | (h) |
| >90 | 60 | 90 | 75 | 90 | 4 | 12 |
| >60-90 | 82.5 | 125 | 82.5 | 105 | 4 | 12 |
| >30-60 | 75 | 112.5 | 82.5 | 105 | 6 | 24 |
| 10-30 | 75 | 112.5 | 82.5 | 105 | 12 | 48 |

*Calculated using Cockcroft-Gault formula
**Recommended starting dose

The Cockcroft-Gault formulas for creatinine clearance (CrCl) are:

CrCl (male)=((140−age)×weight in kg)/(serum creatinine×72)

CrCl (female)=CrCl (male)×0.85

The recommended starting dose of 80 mg is the FDA recommended dosage. A physician can elect to start a patient on a higher dose (e.g., 120 mg), if deemed appropriate.

The minimum delay to first oral dose is the time from the end of the IV infusion to the first oral dose. In embodiments, the delay to the first oral dose is chosen from about 4 hours, 5 hours, 6 hours, 7 hours, 8 hours, 9 hours, 10 hours, 11 hours, or 12 hours.

The oral dosing interval refers to the time between oral dosages. 12 h is B.I.D. (or BID). 24 h is Q.D. (or QD).

In embodiments, the loading dose is delivered via IV infusion over a period of about 1 hour, such as in the range of about 55 minutes to 65 minutes, about 50 minutes to about 70 minutes, or about 45 minutes to about 75 minutes, and is administered/delivered to the patient/subject while admitted to a hospital or other facility capable of providing cardiac resuscitation and continuous electrocardiographic monitoring.

Table 1 shows that the IV loads for initiation of a target dose of 80 mg are 60 mg (>90 mL/min CrCl), 82.5 mg (60-90 mL/min CrCl), and 75 mg (30-60 mL/min CrCl and 10-30 mL/min CrCl). Additional examples of the IV load for the target dose of 80 mg include 49-90 mg, such as 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Table 1 shows that the IV loads for initiation of a target dose of 120 mg are 90 mg (>90 mL/min CrCl), 125 mg (60-90 mL/min CrCl), and 112.5 mg (30-60 mL/min CrCl and 10-30 mL/min CrCl). Additional examples of the IV load for the target dose of 120 mg include 75-135 mg, such as 82-128 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Table 1 shows that the IV loads for escalation from 80 to 120 mg are 75 mg (>90 mL/min CrCl), and 82.5 mg (60-90 mL/min CrCl, 30-60 mL/min CrCl, and 10-30 mL/min CrCl). Additional examples of the IV load for escalation to 120 mg include 63-96 mg, such as 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Table 1 shows that the IV loads for escalation from 120 to 160 mg are 90 mg (>90 mL/min CrCl), and 105 mg (60-90 mL/min CrCl, 30-60 mL/min CrCl, and 10-30 mL/min CrCl). Additional examples of the IV load for escalation to 160 mg include 80-120 mg or 88-140.8 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mg.

In embodiments, the oral dosing begins after the patient/subject has been released from the hospital or other facility that was providing the monitoring, but the patient/subject can still be monitored in a similar way, such as by using a portable/wearable ECG monitoring system. In other embodiments, the patient/subject is not monitored using a portable/wearable ECG monitoring system after release from the hospital or other facility that was providing monitoring. In embodiments, the time of when oral dosing begins depends on the CrCl of the patient. Oral dosing for CrCl of >90 mL/min and 60-90 mL/min typically begins 4 h after completion of the IV infusion (e.g., 5 hours after the start of a 1 h IV). Oral dosing for CrCl of 30-60 mL/min typically begins 6 h after completion of IV infusion (e.g., 7 h after the start of a 1 h IV). Oral dosing for CrCl of 10-30 mL/min typically begins 12 h after IV infusion (e.g., 13 h after the start of a 1 h IV). Additional examples of when the oral dosing begins for a CrCl of >90 mL/min include 2-6 h after completion of infusion. Further examples include 2, 3, 4, 5, to 6 h. Additional examples of when the oral dosing begins for a CrCl of 60-90 mL/min include 2-6 h after completion of infusion. Further examples include 2, 3, 4, 5, to 6 h. Additional examples of when the oral dosing begins for a CrCl of 30-60 mL/min include 4-8 h after completion of infusion. Further examples include 4, 5, 6, 7, to 8 h. Additional examples of when the oral dosing begins for a CrCl of 10-30 mL/min include 10-14 h after completion of infusion. Further examples include 10, 11, 12, 13, to 14 h.

### Example 2

An example sotalol treatment protocol for a patient experiencing AF but who is currently in normal sinus rhythm is described herein. The male patient, age 60, is admitted to initiate treatment in a hospital or other facility capable of providing cardiac resuscitation and continuous electrocardiographic monitoring. The patient's creatinine clearance is determined to be >90 mL/min. The patient is connected to an electrocardiograph and an initial QTc is determined to be less than 450 ms. A physician determines the patient should be initiated on an oral sotalol dosing regimen of 80 mg by mouth twice a day. Treatment is initiated with an IV loading dose of sotalol hydrochloride in an amount of about 60 mg over a period of about 1 hour. The patient's QTc interval is measured every 15 minutes during the IV loading dose administration, along with heart rate and blood pressure. The patient's QTc interval does not exceed 500 ms during any of these measurements, and the ΔQTc is less than 20%. The patient would or is expected to achieve a steady state Cₘₐₓ within about 1-2 hours of administration of the IV sotalol.

Once it is determined that the patient/subject is capable of tolerating the sotalol by way of the IV loading dose, the oral dosing begins after the patient/subject has been released from the hospital or other facility that was providing the monitoring, but the patient/subject can still be monitored in a similar way, such as by using a portable/wearable ECG monitoring system. A first oral dose of 80 mg can be administered to the subject about 5 hours after initiation of the IV loading dose (or about 4 hours after completion of the IV loading dose). The patient's QTc interval is monitored following administration of the first oral dose. The patient's QTc interval still does not exceed 500 ms and the patient's ΔQTc is less than 20%. The patient would achieve a steady state Cₘₐₓ within about 8 hours of administration of the IV sotalol. Subsequent oral doses of 80 mg would be administered by the patient every 12 hours preferably under and using a portable/wearable ECG monitoring system or other monitoring system that can be used at home and/or away from the hospital/facility.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of administering therapy using a single intravenous (IV) dosage of sotalol hydrochloride, the method comprising:
administering to a subject a single IV dosage of sotalol hydrochloride;
wherein the subject is in a facility capable of providing cardiac resuscitation and continuous electrocardiographic monitoring;
wherein the single IV dosage is administered in an amount in the range of about 49.5-128 mg; and
at least about 1 hour after completion of the single IV dosage, and after being discharged from the facility capable of providing cardiac resuscitation and continuous electrocardiographic monitoring, administering an oral dosage of sotalol hydrochloride selected from 80 mg, 120 mg, or 160 mg.

2. The method of claim 1, wherein:
the single IV dosage comprises an amount of sotalol hydrochloride in the range of from about 88.2 mg to 96 mg and is administered to the subject over a period of 1 hour;
the subject has normal kidney function and a creatinine clearance of >90 mL/min; and
the oral dosage of sotalol hydrochloride is 160 mg.

3. The method of claim 1, wherein:
the single IV dosage comprises an amount of sotalol hydrochloride in the range of from about 82 mg to 88 mg and is administered to the subject over a period of 1 hour; and
the oral dosage of sotalol hydrochloride is 80 mg.

4. The method of claim 1, wherein:
the single IV dosage comprises an amount of sotalol hydrochloride in the range of from about 110 mg to 128 mg and is administered to the subject over a period of 1 hour;
the subject is renally impaired and has a creatinine clearance of 10-30 mL/min; and
the oral dosage of sotalol hydrochloride is 120 mg.

5. The method of claim 1, wherein:
the single IV dosage comprises an amount of sotalol hydrochloride in the range of from about 73.8 mg to 82 mg and is administered to the subject over a period of 1 hour;
the subject is renally impaired and has a creatinine clearance of 10-30 mL/min; and
the oral dosage of sotalol hydrochloride is 80 mg.

6. The method of claim 1, wherein:
the subject is being treated for atrial fibrillation and/or atrial flutter, the subject is currently in normal sinus rhythm, and the subject is naïve to sotalol or has not received sotalol for at least five (5) half-lives of sotalol; and
the single IV dosage comprises an amount of sotalol hydrochloride in the range of from about 99 mg to 141 mg.

7. The method of claim 1, wherein the sotalol hydrochloride is administered over a period of time such that the subject is capable of experiencing a sotalol hydrochloride Cₘₐₓ steady state within about 8 hours of administration of the single IV dosage.

8. The method of claim 1, further comprising:
measuring a QT or QTc interval of the subject before administering the single IV dosage;
and measuring a QT or QTc interval of the subject after administering the single IV dosage, but before administering the first oral dose;
wherein the QT or QTc interval after administering the single IV dosage is less than a 20% increase from the QT or QTc interval measured before administering the single IV dosage.

9. The method of claim 1, wherein the subject has a cardiovascular condition selected from atrial fibrillation, atrial flutter, ventricular tachycardia, hemodynamically stable or unstable ventricular tachycardia, paroxysmal atrial fibrillation, ventricular fibrillation, paroxysmal supraventricular tachycardia, heart failure, coronary artery disease, and pulmonary artery hypertension.

10. The method of claim 1, wherein the subject is being treated for atrial fibrillation and/or atrial flutter.

11. The method of claim 10, wherein the subject is currently in normal sinus rhythm.

12. The method of claim 1, wherein the single IV dosage is administered to the subject in an amount and over a period of time such that the sotalol hydrochloride reaches or is capable of reaching Cₘₐₓ steady state in the subject during the administering of the single IV dosage or within 2 hours of the administering of the single IV dosage.

13. The method of claim 1, wherein the single IV dosage is administered to the subject in an amount and over a period of time such that the sotalol hydrochloride reaches or is capable of reaching a Cₘₐₓ in the subject that is at least about 85% of a steady state Cₘₐₓ for an oral dosing protocol of 80 mg, 120 mg, or 160 mg sotalol hydrochloride.

14. A method of initiating or escalating sotalol hydrochloride treatment in a subject comprising:
(A) determining a creatinine clearance of the subject;
(B) determining a QT or QTc interval of the subject;
(C) administering to the subject an IV loading dose of sotalol hydrochloride, wherein the IV loading dose is selected from an amount in the range of from about 49.5 mg to 141 mg;
(D) determining a second QT or QTc interval of the subject; and
(E) providing a prescription for administering oral sotalol hydrochloride to the subject in a manner such that:
a first oral dose of about 80 mg, 120 mg, or 160 mg is to be administered to the subject about 2-12 hours after completion of the IV loading dose; and
one or more subsequent oral dose(s) of about 80 mg, 120 mg, or 160 mg is to be administered to the subject at about 12 hour, 24 hour, or 48 hour interval(s), beginning about 12 hours, 24 hours, or 48 hours after administration of the first oral dose.

15. The method of claim 14, wherein the IV loading dose is administered to the subject in a hospital or facility capable of providing cardiac resuscitation and continuous ECG monitoring.

16. The method of claim 15, wherein the first oral dose and one or more of the subsequent oral dose(s) are administered after the subject is discharged from the hospital or facility capable of providing cardiac resuscitation and continuous ECG monitoring.
